(19) United States
(12) Patent Application Publication
(10) Pub. No.: US 2004/0054377 A1
(43) Pub. Date: Mar. 18, 2004

(54) Flexible cannula

(76) Inventors: Thomas L. Foster, Poland, IN (US); Frederick D. Roemer, Bloomington, IN (US)

Correspondence Address: BRINKS HOFER GILSON & LIONE, P.O. BOX 10395, CHICAGO, IL, 60611, US

(21) Appl. No.: 10/617,580

(22) Filed: Jul. 11, 2003

Related U.S. Application Data
(60) Provisional application No. 60/395,280, filed on Jul. 12, 2002.

(51) Int. Cl. A61B 17/32

(52) U.S. Cl. 606/167; 604/127

(57) ABSTRACT

A flexible cannula is revealed, the cannula useful in graspers for the removal of objects such as stones, calculi, concretions, foreign bodies and the like from the urinary, biliary, vascular or other systems. The flexible cannula is made by removing material in a portion of the cannula near the distal end. The material is preferably removed by laser-cutting the cannula in a spiral pattern, so that material continuity and integrity is maintained, while allowing much greater flexibility. The technique may be used on standard graspers and on laser graspers, and is useful as a part of medical devices, such as ureteroscopes, in which a physician enters a body to remove objects. The flexible cannula may also be used with other devices to cut or spear objects for removal, such as for a biopsy.

This application claims the benefit of the filing date under 35 U.S.C. § 119(e) of Provisional U.S. Patent Application Serial No. 60/395,280, filed on Jul. 12, 2002, which is hereby incorporated by reference in its entirety.

### FIELD OF THE INVENTION

This invention relates generally to surgical devices and more particularly to devices for capturing and retrieving or extracting stones, calculi, concretions, foreign bodies and the like from a human or veterinary patient.

### BACKGROUND OF THE INVENTION

Various organs and passages in the body are subject to the development of stones, calculi and the like. For example, gallstones are a common problem in the United States and are the most frequent cause of gallbladder inflammation. Calculi and concretions in other parts of the biliary system are also commonplace. Similarly, stones, calculi, concretions and the like can develop throughout the renal or urinary system, not only in the ureters and distal to them, but also in the renal tubules and in the major and minor renal calyxes.

Minimally invasive surgical procedures have been developed for the removal of stones, calculi, concretions and the like from the biliary and urinary systems, as well as for the removal or retrieval of foreign bodies from a variety of locations in the body. Such procedures avoid the performance of open surgical procedures such as, for example, a cholecystectomy. Minimally invasive procedures can instead employ percutaneous access, in which stones, calculi, concretions, foreign bodies and the like are removed through a percutaneously inserted access sheath. Several access routes are suitable, depending upon the specific system and the particular location in the system at which the stones, calculi, concretions, foreign bodies or the like are found. One access route that is frequently used is the urethra.

Without regard to the particular access route, percutaneous extraction is often based upon the use of catheters or similar devices to engage and remove the stones, calculi, concretions, foreign bodies and the like. Such catheters and devices typically comprise a hollow, flexible sheath and a plurality of wires positioned in but extendable from the sheath. The wires are joined or arranged so as to form a means, such as a basket or forceps for engaging the object to be retrieved when the wires are extended from the sheath. The wires may also form a continuum with the sheath. The engagement means (for example, a basket) can be collapsed by withdrawing the wires into the sheath. A helical basket permits entry of the stone or the like from the side of the basket, while an open ended (“eggwhip”) basket allows a head-on approach to the stone or the like. Other retrievers and graspers can include forceps or can include a loop or snare for encircling the body to be removed, the loop or snare being made of the wire.

Despite their successful use for some time, such retrieval devices are subject to drawbacks. For example, in a typical ureteroscope, the retrieval device is only one of several devices that are used to enter, irrigate, inspect, grasp, break up, and then remove the stones or calculi discussed above. Thus, the ureteroscope tends to be relatively thick, even when individual components are of small diameter. It would be desirable to make the individual components of smaller diameter, but it would also be desirable to make the components more flexible.

When the ureteroscope or extraction device portion thereof leaves the ureter and enters the kidney, it is necessary to go in a forward direction for stones or objects ahead. It would be very helpful for an extraction device to have an ability to bend backward to at least inspect portions of the kidney that are behind the device. A grasper that is very flexible and could bend backwards would be very helpful to the surgeon removing kidney stones. If the grasping device is equipped with a laser fiber for breaking up objects as well as retrieving and removing them, its flexibility may be even further limited by the larger diameter required for the laser fiber itself, as well as its outer sheath.

It would be highly desirable to have a device for use inside the human body for the capture and retrieval or extraction of stones, calculi, concretions, foreign bodies and the like which has much greater flexibility than present devices, and which is capable of bending back upon itself, or capable of bending around a small diameter, such as ¼″ or ½″ (about 6 mm or 13 mm).

### BRIEF SUMMARY OF THE INVENTION

The foregoing problems are solved and a technical advance is achieved in a flexible cannula useful in an extraction or retrieval device for capturing and extracting, retrieving or removing objects such as stones, calculi, concretions, foreign bodies and the like from a variety of locations in the body. The flexible cannula may also be used for removing tissue from a body, such as for a biopsy sample. Of course, the device is not limited to human bodies, but may also be used in veterinary applications. One embodiment is a flexible cannula, the cannula comprising a proximal portion, and a distal portion which comprises a spiral cut along a longitudinal axis of the cannula. The cannula is preferably a continuum, a single continuous piece of material, preferably stainless steel or Nitinol. Other shape-memory alloys may also be used, but this particular nickel-titanium alloy is preferred. The cuts made in the cannula are preferably as narrow as possible, with cut widths from about 0.001 inches to about 0.002 inches being preferred, although wider cuts may also be used and are easier to manufacture. The cannula is preferably made from a hollow tube of small diameter.

Another embodiment of the invention is a grasper at a distal end of the cannula, the grasper forming a continuum with the flexible cannula. The grasper may comprise a sheath enclosing at least a portion of the flexible cannula. Another embodiment is a flexible cannula comprising a proximal portion and a distal portion which comprises a spiral cut. The spiral cut is taken from about sixty degrees to about eighty degrees from a longitudinal axis of the cannula, and wherein the proximal portion and the flexible portion comprise a continuum of metal. The flexible cannula also comprises a first intermediate portion between the distal portion and proximal portion.

Another embodiment of the invention is a method of extracting an object. The method comprises placing a grasper near an object, and grasping the object. The method then comprises a step of removing the object, wherein the grasper has been made from a cannula, the cannula having a distal portion which comprises a spiral cut from a longitudinal axis of the cannula. There are many other embodiments of the invention, which will be made clearer in the accompanying drawings and description.

### BRIEF DESCRIPTION OF SEVERAL VIEWS OF THE DRAWINGS

The present invention will now be described in conjunction with the following drawings, wherein like reference characters refer to like parts throughout the several views.

FIG. 1 is a plan view of a first embodiment.

FIG. 2 is a plan view of a second embodiment.

FIGS. 3a and 3b are plan views of a third embodiment and closer view of the grasper portion of the embodiment.

FIG. 4a is a plan view of a grasper device using the embodiment of FIGS. 3a and 3b.

FIG. 4b is an end perspective view of the grasper device of FIG. 4a.

FIG. 5 is a plan view of a laser grasper using the embodiment of FIG. 2.

FIG. 6 is a cross-sectional view of a ureteroscope using a flexible cannula.

FIGS. 7-9 are plan views of cannulae made for removing an object from a body.

FIGS. 10-13 are plan view of other embodiments of cannulae according to the present invention.

FIG. 14 is a method of extracting an object.

### DETAILED DESCRIPTION OF THE PRESENTLY PREFERRED EMBODIMENTS

One embodiment of the invention is a flexible cannula made from a single continuous piece of tubing, as depicted in FIG. 1. The flexible cannula 10 comprises a hollow tube with a proximal portion 12, a first distal portion 14, and a second distal portion 17. The first distal portion is made flexible by removing material from the wall of the tube, preferably in a spiral pattern 16, as shown. The spiral pattern 16 forms an angle with a longitudinal axis 18 of the cannula. The angle formed is preferably from about sixty degrees to about eighty degrees, wherein a ninety degree angle is at a right-angle to the longitudinal axis of the cannula. The cannula may be made from solid rod or from hollow tubing, but tubing is preferred because at least one lumen 19 through the center is desired. Tubing with outer diameters from 0.022 inches and larger are preferred, included tubing with an inner diameter/outer diameter of 0.019/0.027 and 0.026/0.034. Those with skill in the art will recognize that tubes with larger diameters tend to be easier to process, while tubes with smaller diameters are preferred for medical applications.

The spiral cut is preferably tightly controlled, with a width of cut preferably from about 0.001 to about 0.002 inches. Wider cuts are more easily made, but smaller widths allow the cannula to retain more strength while achieving flexibility. The cuts are preferably made with a laser cutter, although cuts may also be made by other methods, including chip-cutting type machining and water-jet cutting. The spiral cut allows the continuum of material that is the cannula to have a desirable degree of flexibility, while retaining the small size and diameter possible with a single piece of material. A continuum in which there are no welds or joints takes up very little valuable space within the lumen of a laser grasper or retrieval instrument. In some embodiments, cannulae made with spiral-cut sections can effortlessly bend around steel rods with ¼″ and ½″ diameter (about 6 mm and 13 mm respectively). They can therefore bend back upon themselves, enabling a much greater range by a clinician or surgeon employing a grasper made from such a cannula.

The cannula 10 may be composed of any medical grade material having strength suitable for introduction to the site from which an object is to be retrieved, and having a configuration designed for secure grasping, containment and/or removal of the object. The cannula is preferably composed of a metal such as stainless steel or Nitinol (the latter being preferably in a superelastic state). However, the cannula may also be composed of synthetic materials of suitable strength, such as polymeric or plastic materials having fibrous or particulate fillers incorporated in them, especially if the cannula or a retrieval device made from the cannula is to be used for purposes other than a medical operation on a human being. It should be noted that polymeric and plastic materials lacking such fillers are generally less preferred embodiments of the invention, because such materials lack the strength necessary to function adequately in the range of diameters preferred in the practice of the present invention. This is believed to be true even of relatively strong and stiff materials, such as the polyimides and polyamides. Specific materials of potential use include, but are not limited to, nylons, polycarbonates, polytetrafluoroethylene, and any other reinforced or un-reinforced plastic material suitable for the application.

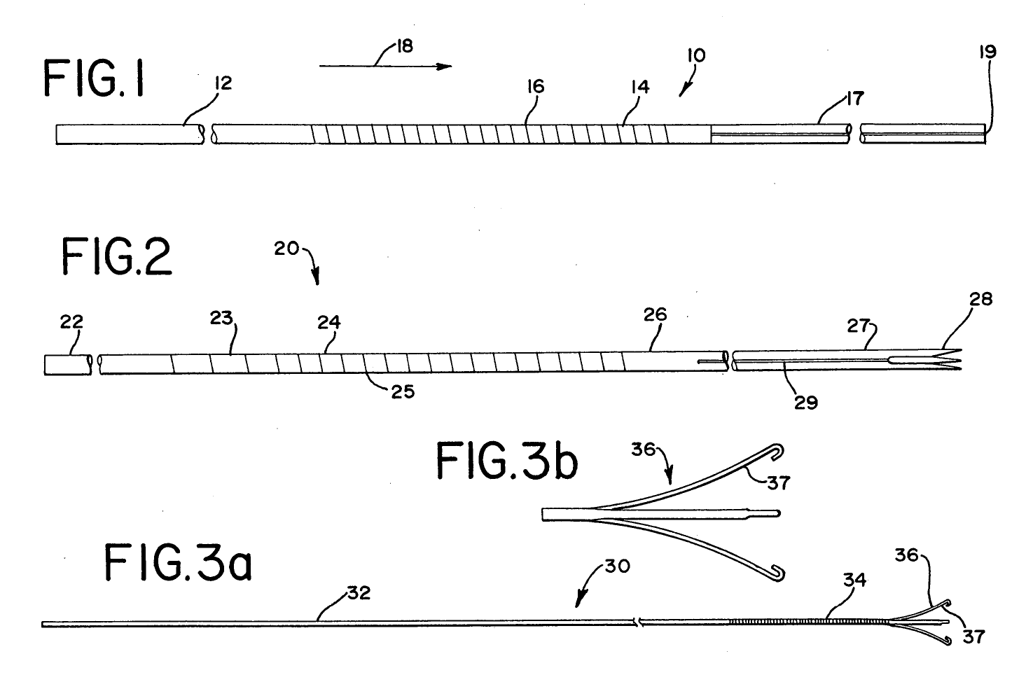

The flexible cannula may also comprise a grasping portion of a grasping instrument. FIG. 2 illustrates a cannula 20 having a proximal portion 22, a first distal portion 24 which comprises a spiral cut, an intermediate portion 26 that is not spiral cut, a second distal portion 27, a grasper portion 28, and a central lumen 29. The cannula may comprise a hollow tube of the same material and size discussed above, and the first distal portion 24 may also have material cut in a spiral pattern 25 as shown in the figure. In one embodiment, the overall length of the cannula is from about four feet to about five feet (about 1.2 m to about 1.5 m), with a preferred length of about fifty-one inches (about 1.3 m). The first distal portion may have material removed in a spiral cut at an angle to a longitudinal axis of the cannula of from about sixty degrees to about eighty degrees. A flexible cannula according to the present invention may have more than one portion having a spiral cut, such as a first spiral-cut portion, an intermediate portion, and a second spiral cut portion. A second intermediate portion may then be interposed between the final spiral-cut portion and a tool or grasper at the end of the cannula.

The precise angle and length of the flexible section are not essential, but the resulting section should have flexibility and strength sufficient for the desired application. Thus, in one embodiment, the section which comprises a spiral cut is about three inches long (77 mm long) and has a spiral cut at an angle of about seventy-five degrees from a longitudinal axis of the cannula. The width of the spiral cut is preferably about 0.001 to about 0.002 inches (about 0.025 to about 0.05 mm). The cannula with a grasping portion may also have an intermediate portion 26 between the first distal portion 24 and the second distal portion 27. The intermediate portion 26 may be only a short portion of the cannula, as little as 0.1 inches (2.5 mm) to 1.0 inches (25 mm). The second distal portion may also be relatively short, from about 0.5 inches (about 13 mm) to about 1.0 inch (25 mm). The end of the cannula may comprise a grasping portion 28, in which the cannula is split into three or more arms or tongs for grasping an object. The grasping portion may be as long as 0.25″ (about 6 mm) or longer, depending on the application needed. In one embodiment, the grasping portion comprises as little as about 0.1 inches (2.5 mm) of length.

The cannula with a grasping portion may also comprise a second intermediate portion 23 between the proximal portion 22 of the cannula 20 and the first distal portion 24. The second intermediate portion 23 may comprise from about 0.5 inches (13 mm) to about 2 inches (51 mm) of length of the cannula. The second intermediate portion may be useful in imparting a smaller degree of flexibility to the cannula than the first distal portion 24. The second intermediate portion 23 has a spiral cut also. This spiral cut may be only one-sixth to one-third as long as the first distal portion, and may also have a much larger pitch in its helical cut. Pitch is defined as the axial distance between corresponding points in the helical cut on the outer diameter of the cannula. Thus, in one embodiment, the first distal portion 24 may have a pitch of about 0.021 inches (about 0.5 mm). The second intermediate portion 23 may have a pitch of 0.04 inches (about 1 mm). The pitch of this portion is not limited to a constant value, but may vary as desired to achieve a desired degree of flexibility. In one embodiment, intermediate portion 23 may have an exponentially decreasing pitch, in which the pitch begins at a large value, as much as five times the pitch in the flexible portion 25, and exponentially decreases over several turns, until the pitch reaches the pitch value of the first distal portion. Any pitch may be used that yields a desirable degree of flexibility in this portion of the cannula.

The cannula with a first distal portion and a grasper portion may be used in a grasper for use inside the body of a human being. Other applications may be used for veterinary applications, or other applications in which a flexible grasper may be useful, such as mechanical or hydraulic applications. A flexible cannula 30 with a grasper is depicted in FIG. 3a. The cannula 30 has a proximal portion 32, a distal portion 34 and a grasper portion 36. In a preferred embodiment, the grasper portion 36 is about 0.1 inches long (about 2.5 mm) and is formed by removing material from the cannula to form three grasper arms. The cannula with grasper may be heat treated or otherwise processed so that when the arms 37 are unrestrained by a sheath or other member, the arms are separated by about 0.40 inches (about 10 mm). A closer view of the grasper portion 36 and arms 37 appears in FIG. 3b. The grasper arms 37 form a continuum with the grasper 36, the distal portion 34 and the proximal portion 32.

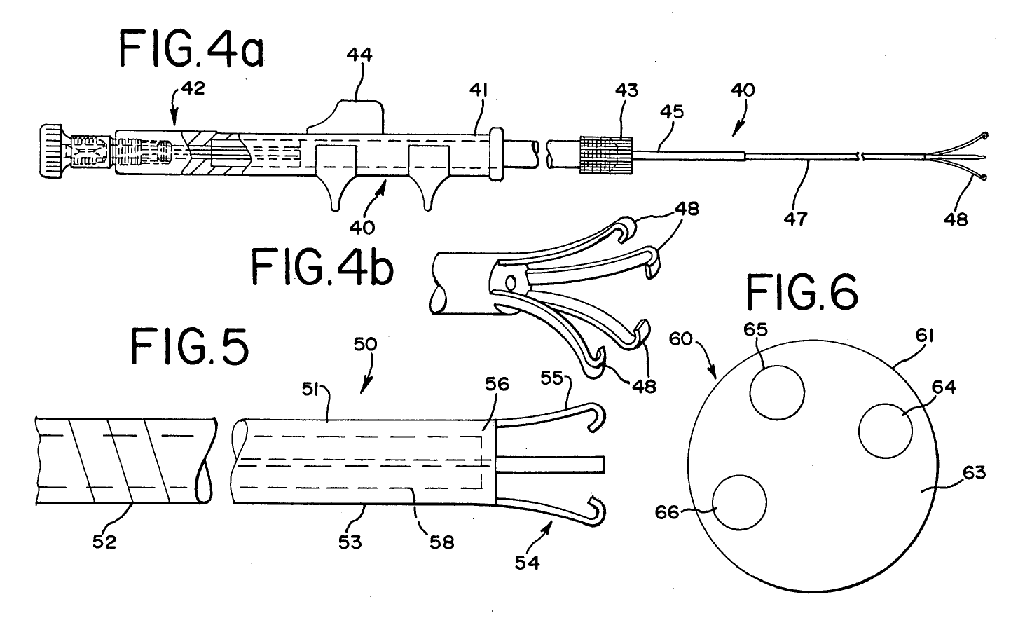

A grasper 40 may use the flexible cannula 47 in retrieving objects. As shown in FIG. 4a, the grasper comprises a handle 41 with a collet mechanism 42. The operating handle 44 is connected to flexible cannula 47 for extending or retracting the cannula and grasper portion 48. A sheath 45 that contains the flexible cannula 47 may be connected via sealing connector 43. In operation, the surgeon places the cannula near an object and extends or retracts the cannula 47 to retrieve objects with the grasper 48. Sheath 45 is desirably larger in diameter than the outer diameter of the flexible cannula, so that the cannula can be easily extended from and retracted into the sheath. An end perspective view of the grasper of FIG. 4a is shown in FIG. 4b, depicting the grasper 48 with four arms 49.

The flexible cannula of the present device may be used for other applications as well. A highly flexible cannula may be used in a laser grasper, in which the central lumen of the device includes a laser carrier for breaking up stones or calculi, such as kidney stones. An application with such a laser is depicted in FIG. 5, in which a laser grasper 50 comprises a flexible cannula 51 with a first distal portion 52 and a second distal portion 53 with a grasper portion 54 and grasper arms 55. The grasper has a central lumen 56 in which a laser carrier 58 resides, the laser carrier useful for breaking up stones and calculi within the body of a patient. As is well known in the art, a laser carrier with a 200μ laser fiber has an actual diameter of about 0.015-0.016 inches (about 0.38-0.41 mm). One embodiment of the invention is a laser grasper having a 200μ shielded laser carrier within a flexible cannula having an inner diameter of about 0.019 inches (0.5 mm, about 1.5 Fr) and an outer diameter of about 0.027 inches (0.7 mm, about 2.1 Fr), and using a sheath of about 0.0345 inches outer diameter (about 0.88 mm).

It should be clear from the foregoing that the flexible cannula of the present invention is particularly advantageous over prior devices in a variety of ways. Most importantly, the present invention is advantageous over the prior art in that the device has a great deal of flexibility in allowing a surgeon to remove undesirable objects from a body. In a laser grasper, a surgeon may survey the operating field and deploy the laser to reduce the size of stones and other objects. The surgeon may also enjoy many degrees of freedom in collecting and removing the resulting particles. Another application for the flexible cannula, a ureteroscope 60 is depicted in FIG. 6. A distal portion of the ureteroscope may include an outer flexible cannula 61 having a central lumen 63. Within the central lumen may reside an optical fiber 64 for visualizing the operating field, an irrigation port 65, and a laser carrier 66, for the surgeon to deploy to break up stones and other undesirable particles within a body. The flexible cannula may also possess a distal portion with a grasper and arms for retrieval and removal of the particles.

The cannula may take on different configurations and may be used for somewhat different purposes than for removing stones and calculi. Devices made from a flexible cannula may be used for removing tissue samples, such as for biopsy purposes as well, in which tissue is cut or removed from the body of a patient. Such devices may be used for entering virtually any bodily cavity, and particularly bodily cavities for which major surgery may otherwise be required. Examples of areas where such devices are desirably used include the vascular system, biliary system, and the genito-urinary system. Flexible cannulae may also be used to remove samples from the colon and from the gastro-intestinal system, and from the lung and throat systems.

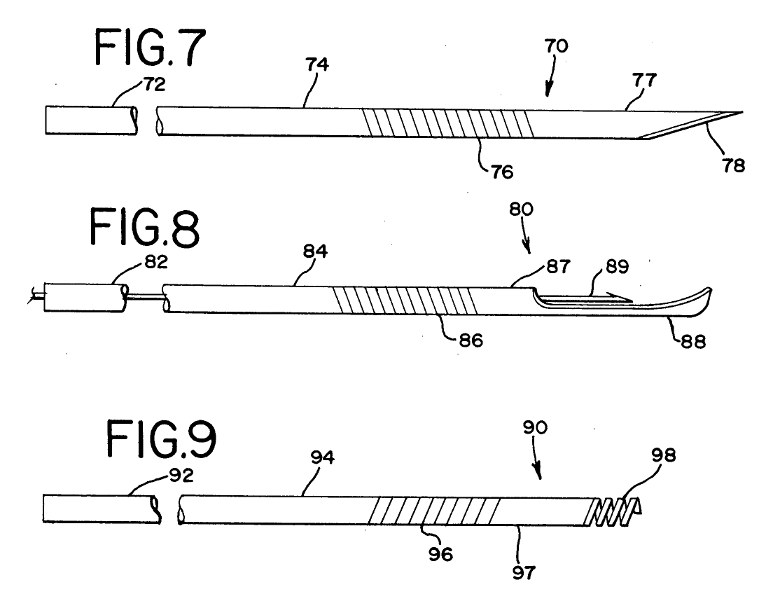

FIGS. 7-9 depict alternative embodiments, each having a different tool or device at a distal end of the flexible cannula. In each of FIGS. 7-9, the device is configured for removal of tissue from a patient, such as a human or a veterinary patient. The cannulae depicted in FIGS. 7-9 each form a continuum of metal that includes the cannula and the device or tool at the distal end of the cannula. FIG. 7 depicts a needle, such as a flexible biopsy needle, with a blade at a distal end for cutting tissue from a patient. The flexible needle 70 comprises a proximal end 72, and a distal end 78 configured as a curved blade. The needle also comprises an intermediate portion 74 and a spiral-cut portion 76, as described above. There is also a second intermediate portion 77 without spiral cuts between the spiral-cut portion 76 and the blade 78 at the distal end. The intermediate portion 77 may be from about 0.010 to about 0.050 inches long (about 0.25 to about 1.25 mm long).

Another embodiment is a flexible cannula having a slicing-type blade at the distal end of the cannula, such as a flexible biopsy blade. This embodiment 80 is shown in FIG. 8. The flexible blade 80 has a proximal end 82 and a distal end with a slicing-type curved blade 88. The blade also comprises an intermediate portion 84 and a spiral-cut portion 86, as described above. There is also a second intermediate portion 87 without spiral cuts between the spiral-cut portion 86 and the slicing-type blade 88 at the distal end. The intermediate portion 87 may be from about 0.050 to about 0.100 inches long. The flexible biopsy blade may also have a barbed stylet 89 running through the center of the blade for retaining a severed sample or severed biopsy material. The stylet or barb may be from about 0.015 to about 0.025 inches in diameter (about 0.38 to about 0.64 mm).

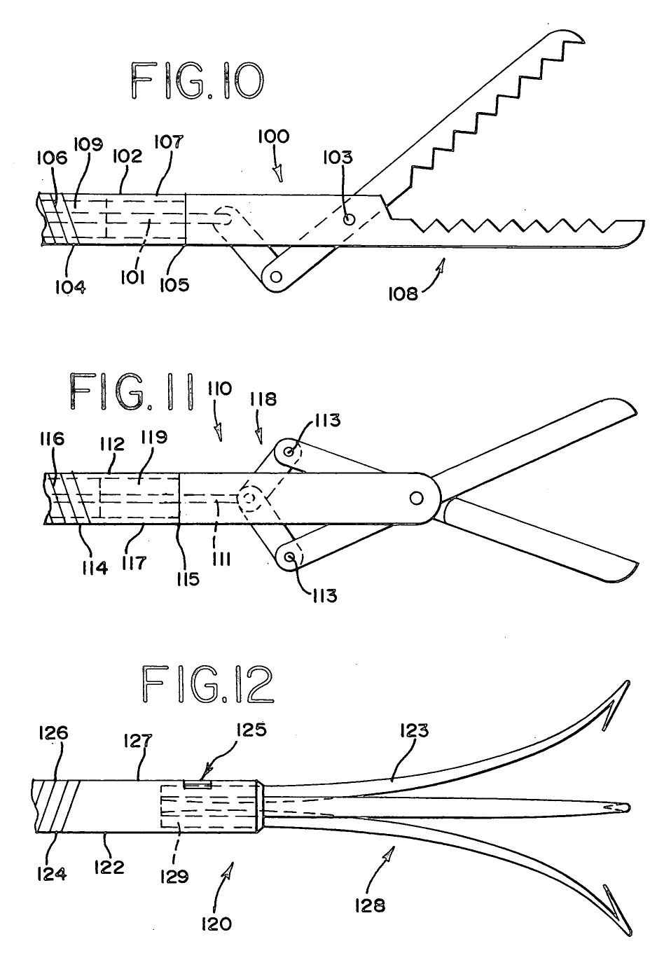

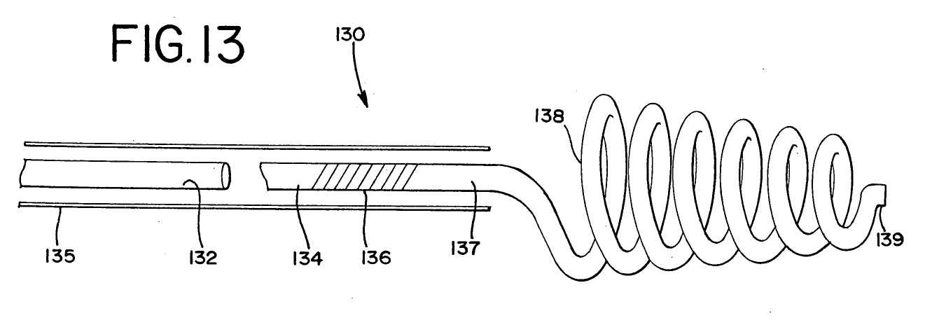

Yet another embodiment is a flexible coring biopsy cannula with a spiral-cut distal end. This embodiment is depicted in FIG. 9. The flexible coring cannula 90 includes a proximal portion 92 and a distal portion 98. The flexible coring cannula includes an intermediate portion 94, a spiral cut portion 96 and a second intermediate portion 97 between the spiral-cut portion 96 and the distal portion 98. The intermediate portion 97 may be from about 0.010 to about 0.050 inches long (about 0.25 to about 1.25 mm). The distal portion 98 is formed by removing material from the distal end of the cannula in the same spiral-cut manner used to form the flexible portion. The distal portion, however, need only extend for a few turns, and may be only from about 0.10 to about 0.20 inches long (about 2.5 to 5 mm long). The coils at the distal end may each be from about 0.030 to about 0.060 inches long (about 0.75 to about 1.5 mm) with gaps from about 0.010 to about 0.050 inches long (about 0.25 to about 1.25 mm) between coils. This configuration makes it easier for a clinician operating the spiral coil device to “spear” an object for removal. Other lengths and configurations are also possible.

The above embodiments, FIGS. 1-9, are preferred because each comprises a single continuum of metal. Other embodiments are also possible using the flexible cannula and attachments. For instance, a grasper device may be welded or soldered onto the cannula. FIG. 10 depicts one such device, a grasper 100 that includes a flexible cannula 102 and a grasper portion 108 that is joined to the cannula 102 with a transition section 105. The transition section may be a weld joint, a solder joint, an interference fit, or other attachment section. Grasper 100 includes a central lumen 109, a proximal section (not shown) and a distal section 107 to which grasper portion 108 is attached. Grasper 100 also has a spiral-cut section 104 made flexible by a helical cut 106 of material removed from the cannula. Grasper 100 is operated by an actuating wire 101 through the central lumen 109 and attached to jaws of grasper portion 108, pivoting on pivot pin 103.

Another device potentially useful in biopsies is depicted in FIG. 11. This is a scissors-type biopsy cannula 110, for cutting and removing an object from a patient. The embodiment comprises a flexible cannula 112 and a scissors-type grasper 118 that is joined to the cannula 112 with a transition section 115. The transition section 115 may be a weld joint or a solder joint, or other attachment section. Biopsy cannula 110 includes a central lumen 119, a proximal section (not shown) and a distal section 117 to which grasper 118 comprising two jaws is attached. The cannula also has a spiral-cut section 114 made flexible by a helical cut 116 of material removed from the cannula. Grasper 110 is operated by an actuating wire 111 through the central lumen 119 and attached to grasper 118, pivoting on pivot pins 113.

FIG. 12 depicts another device useful in biopsies and other applications, a grasper 120 that includes a flexible cannula 122 and a grasper portion 128 that is joined to the cannula 122 with a transition section 125. The transition section may be a weld joint, a solder joint, a snap fit, or other attachment section. Grasper 120 includes a central lumen 129, a proximal section (not shown) and a distal section 127 to which grasper portion 128 is attached. The cannula also has a spiral-cut section 124 made flexible by a helical cut 126 of material removed from the cannula. Operation of the grasper portion 128 and arms 123 is similar to that of the grasper of FIGS. 3a and 3b, with the exception that the grasper portion 128 does not form a continuum of metal with the cannula. Interference fits or snap fits may also be used for attaching any of the graspers to the flexible cannula.

FIG. 13 depicts another embodiment of a useful device made from the flexible cannula previously described. A flexible spiral catcher/extractor 130 is made from a flexible cannula 132 having a distal portion 134 with a helical cut portion 136 and a spiral catcher/extractor 138 at the distal end 139. As described above, there may be a transition portion 137 between the portion having helical cuts 136 and the catcher/extractor 138. In other embodiments, the catcher/extractor may have spiral cuts in the transition portion 137 or in the catcher/extractor portion 138, or in both the transition portion 137 and the catcher/extractor portion 138. The catcher/extractor is not attached or welded to the cannula, but is integral with the length of the cannula, thus allowing for a more reliable structure and easier manufacture. The cannula and spiral catcher/extractor form a single continuum of metal and are desirably made from nitinol or other super-elastic alloy. The cannula may be used with a sheath 135, separate from the cannula.

In operation, a stone, a calculus, or other undesirable object is captured by the spiral catcher positioned at the site of the stone(s) or above the site of the stone(s). The cannula is withdrawn for some length into the sheath. This causes the spiral configuration of the super-elastic alloy to temporarily “shrink”, thus capturing the object and enabling the surgeon or operator to remove the object from a patient's body. Conversely, the spiral catcher may be withdrawn without using the sheath, thus removing the object or the stone(s). The sheath may be made from a number of metallic or plastic materials. Preferred is a thin polyimide sheath, with or without a thin stainless steel inner braid and with or without a PTFE (polythetrafluoroethylene) inner layer.

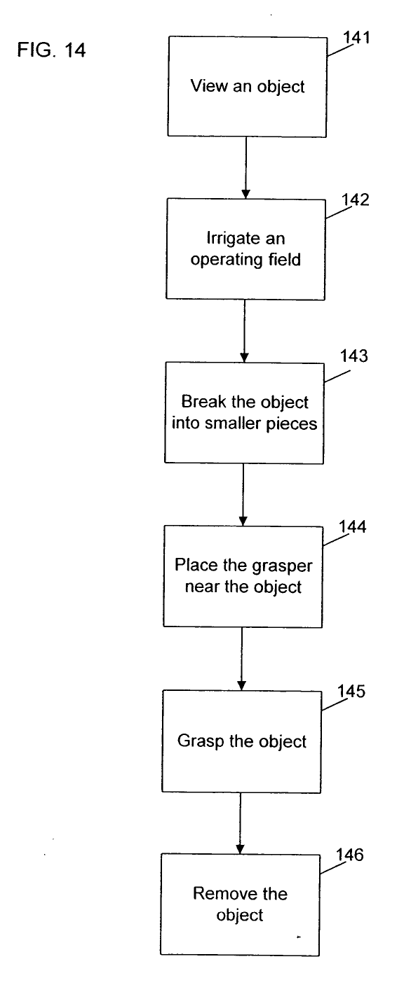

Another embodiment is a method for removing an object from the body of a patient, or extracting an object. FIG. 14 depicts steps of the method. One step 144 of the method is to place a grasper near the object. The grasper is preferably made from a cannula, the cannula having a distal portion which comprises a spiral cut from a longitudinal axis of the cannula. As mentioned above, the cannula and the resulting grasper preferably have very small outer diameters, such as from about 0.022 inches to about 0.039 inches (about 0.5 mm to about 1 mm). Other steps in the method may include grasping the object 145 and removing the object 146 to a desired location, often outside the body of the patient. If the grasper is a laser grasper, the grasper may include a fiber optic device and an irrigation system, and the method may include steps of viewing the object 141, irrigating the operating field 142, and breaking the object into smaller pieces 143.

As noted above, the flexible cannula is expected to find use in a wide variety of procedures, including urological procedures, biliary procedures, vascular procedures and procedures for the retrieval of foreign objects from a variety of body cavities. The details of the construction or composition of the various elements of the flexible cannula and devices using the flexible cannula not otherwise disclosed are not believed to be critical to the achievement of the advantages of the present invention, so long as the elements possess the strength or flexibility needed for them to perform as desired. The selection of such details of construction are believed to be well within the ability of one having skill in the art, in view of the present disclosure.

What is claimed is:

1. A flexible cannula, comprising:
a proximal portion; and
a distal portion which comprises a spiral cut along a longitudinal axis of the cannula, wherein the cannula forms a continuum of material.

2. The flexible cannula of claim 1, wherein the spiral cut is taken at about sixty to about eighty degrees from the longitudinal axis of the cannula, and is about 0.001 to about 0.002 inches (about 0.25 to about 0.5 mm) wide.

3. The flexible cannula of claim 1, wherein the cannula and the continuum further comprise a grasping portion.

4. The flexible cannula of claim 1, wherein the cannula and the continuum further comprise a grasping portion and an intermediate portion between the grasping portion and the distal portion.

5. The flexible cannula of claim 1, wherein the cannula and the continuum further comprise an intermediate portion about 0.5 inches long to about 2 inches (about 13 mm to about 51 mm) long, proximal to the distal portion, having a spiral cut with a pitch less than a pitch of the distal portion.

6. The flexible cannula of claim 1, wherein an outer diameter of the cannula is from about 0.022 inches (0.56 mm, 1.7 Fr) to about 0.034 inches (0.86 mm, 2.6 Fr).

7. A grasper comprising the flexible cannula of claim 1 and a grasper portion at a distal end of the grasper, the grasper portion forming a continuum with the flexible cannula, and also comprising a sheath enclosing at least a portion of the flexible cannula.

8. The flexible cannula of claim 1, further comprising a device at a distal end of the cannula for removing material, the device selected from the group consisting of a blade, a slicing-type blade, a slicing-type blade and a separate barbed stylet through a center of the cannula, a helix, and a helical catcher/extractor, wherein the cannula and the device for removing material form a continuum.

9. A laser grasper, comprising the flexible cannula of claim 1 and a grasper portion at a distal end of the grasper, the grasper and cannula forming a continuum, and also comprising a laser fiber within the cannula.

10. A ureteroscope comprising an optical fiber, an irrigation system, and a flexible cannula according to claim 1.

11. A combination of the flexible cannula of claim 1 and a grasper, wherein the grasper is not a part of the continuum.

12. The flexible cannula of claim 1, wherein the cannula comprises a material selected from the group consisting of stainless steel, Nitinol, a shape-memory alloy, a polymer, and a polymer composite.

13. A flexible cannula, comprising:
a proximal portion;
a distal portion which comprises a spiral cut, wherein the spiral cut is taken at an angle of from about sixty degrees to about eighty degrees from a longitudinal axis of the cannula, wherein the proximal portion and the distal portion comprise a continuum of metal; and
a first intermediate portion between the proximal portion and the distal portion.

14. The flexible cannula of claim 13, wherein the spiral cut is about 0.001 to 0.002 inches wide.

15. The flexible cannula of claim 13, wherein the proximal portion is from about 3 feet long to about 5 feet long and a portion with a spiral cut is from about 2 inches long to about 6 inches long (about 7.5 cm to about 15 cm long).

16. The flexible cannula of claim 13, wherein the cannula and the continuum further comprise a grasping portion.

17. A grasper comprising the flexible cannula of claim 13 and a grasper portion at a distal end of the grasper, the grasper portion forming a continuum with the flexible cannula, and also comprising a sheath enclosing at least a portion of the flexible cannula.

18. A laser grasper, comprising the flexible cannula of claim 13 and a grasper portion at a distal end of the grasper, the grasper and cannula forming a continuum, and also comprising a laser fiber within the cannula.

19. The flexible cannula of claim 13, further comprising a second intermediate portion between a portion with a spiral cut and the grasping portion.

20. The flexible cannula of claim 19, wherein the second intermediate portion has material removed in a spiral cut with a pitch less than a pitch of the distal portion.

21. The flexible cannula of claim 13, further comprising a device for removing material selected from the group consisting of a blade, a slicing-type blade, a slicing-type blade and a separate barbed stylet through a center of the cannula, a helical catcher/extractor, and a helix.

22. A combination of the flexible cannula of claim 13 and a grasper, wherein the grasper is not part of the continuum.

23. A method of extracting an object, the method comprising:
placing a grasper near the object;
grasping the object; and
removing the object, wherein the grasper has been made from a cannula, the cannula having a distal portion which comprises a spiral cut from a longitudinal axis of the cannula.

24. The method of claim 23, wherein the grasper is a laser grasper and further comprising breaking the object into small pieces.

25. The method of claim 23, wherein the grasper further comprises an optical device and a irrigation system, and further comprising irrigating and viewing the object.
